(19) United States
(12) Patent Application Publication
(10) Pub. No.: US 2007/0004623 A1
(43) Pub. Date: Jan. 4, 2007

(54) Use of hydroxylated amino acids for treating diabetes

(75) Inventors: Francesco Bellini, Ville Mont-Royal (CA); Claude Vezeau, Lorraine (CA); Gerard Ribes, Montpellier (FR); Nicolas Chapal, Montreal (CA); Mark Prentki, Ville Mont-Royal (CA)

Correspondence Address: CLARK & ELBING LLP, 101 FEDERAL STREET, BOSTON, MA, 02110, US

(73) Assignee: Innodia Inc., Laval (CA)

(21) Appl. No.: 10/577,512

(22) PCT Filed: Oct. 27, 2004
(86) PCT No.: PCT/CA04/01883

§ 371 Date: Jul. 10, 2006

Related U.S. Application Data
(60) Provisional application No. 60/514,738, filed on Oct. 27, 2003.

(51) Int. Cl. A61K 38/22; A61K 31/426; A61K 31/198; A61K 31/17; A61K 31/155

(52) U.S. Cl. 514/12; 514/369; 514/561; 514/594; 514/635

(57) ABSTRACT

This invention relates to methods and compositions for treating diabetes, which involve the use of hydroxylated amino acids, such as 4-hydroxyisoleucine, and one or more additional antidiabetic agents.

### FIELD OF THE INVENTION

This invention relates to methods and compositions for use in treating diabetes.

### BACKGROUND OF THE INVENTION

Diabetes mellitus is a disorder of carbohydrate metabolism, and develops when the body cannot effectively control blood glucose levels. The disease is characterized by inadequate secretion or utilization of insulin, high glucose levels in the blood and urine, and excessive thirst, hunger, weight loss, and urine production. It can lead to a number of serious complications, including cardiovascular disease, kidney disease, blindness, nerve damage, and limb ischemia.

Diabetes is divided into two types, 1 and 2, with the latter accounting for about 90% of cases. In type 1 diabetes, the body destroys the insulin-producing β cells of the pancreas, resulting in the inability of the body to produce insulin. Type 1 diabetes typically occurs in children or young adults, and generally is managed by insulin administration, strict diet, and exercise. Type 1 diabetes is observed as well in older adults following therapeutic failure of type 2 diabetes. Type 2 diabetes is characterized by impaired insulin secretion due to altered β cell function, as well as decreased ability of normally insulin sensitive tissues (e.g., the liver and muscle) to respond to insulin. Type 2 diabetes generally develops in those over 45, but is recently also being detected in younger people. The disease is associated with risk factors such as age, family history, obesity, lack of regular exercise, high blood pressure, and hyperlipidemia. Treatment involves strict diet and exercise regimens, oral medications (e.g., medications that increase insulin secretion and/or insulin sensitivity), and, in some cases, insulin administration.

Type 2 diabetes is rapidly increasing in its importance as a major public health concern in the Western world. While one hundred years ago it was a relatively rare disease, today there are about 200 million type 2 diabetics worldwide, and this number is estimated to increase to greater than about 300 million by the year 2025. This dramatic increase in the incidence of type 2 diabetes parallels an increase in the prevalence of obesity in Western cultures. Further, as more cultures adopt Western dietary habits, it is likely that type 2 diabetes will reach epidemic proportions throughout the world. Given the seriousness of the complications associated with this disease, as well as its rapidly increasing incidence, the development of effective approaches to treatment is a primary concern in the field of medicine.

### SUMMARY OF THE INVENTION

The invention provides methods of treating diabetes (type 1 diabetes or type 2 diabetes) in patients, which involve administering to the patients a hydroxylated amino acid (for example, 4-hydroxyisoleucine, e.g., the 2S,3R,4S isomer of 4-hydroxyisoleucine) and one or more additional antidiabetic agents, to obtain an improved (e.g., synergistic or additive) effect. Examples of additional antidiabetic agents that can be used in the invention include biguanides (e.g., metformin), sulfonylurea drugs, glinides, glitazones (e.g., thiazolidinediones, such as rosiglitazone maleate), glucagon-like peptide 1 receptor agonists (e.g., Exenatide®), and insulin. Other examples of antidiabetic (and other) agents that can be used in combination with hydroxylated amino acids according to the invention are listed below. In one example, 4-hydroxyisoleucine is combined with insulin and/or metformiin, while in another example, 4-hydroxyisoleucine is combined with metformin and/or a thiazolidinedione. The hydroxylated amino acid and other antidiabetic agents can be administered at or about the same time as one another or at different times. Also included in the invention are pharmaceutical kits and compositions (e.g., tablets or capsules) that include combinations of the agents noted above and elsewhere herein.

The invention provides several advantages. For example, because the drug combinations described herein are used to obtain improved (e.g., synergistic or additive) effects, it is possible to consider administering less of each drug, leading to a decrease in the overall exposure of patients to drugs, as well as any untoward side effects of any of the drugs. In addition, greater control of the disease may be achieved, because the drugs can combat the disease through different mechanisms.

Other features and advantages of the invention will be apparent from the following detailed description and the claims.

### BRIEF DESCRIPTION OF THE DRAWINGS

FIG. 1 is a graph showing additive stimulation of glucose uptake in 3T3-L1 differentiated adipocytes by the combination of insulin and ID 1101.

FIG. 2 is a series of graphs showing changes in plasma glucose levels from baseline during an oral glucose tolerance test.

FIG. 3 is a graph showing the effect of ID 1101 in combination with Glibenclamide on insulin secretion in INS-1 beta cells.

FIG. 4 is a graph showing the effect of ID 1101 in combination with Exendin-4 on insulin secretion in INS-1 beta cells.

### DETAILED DESCRIPTION OF THE INVENTION

The invention provides methods and pharmaceutical kits or compositions for use in treating diabetes and related diseases or conditions, such as metabolic syndrome. The invention is based on the administration of hydroxylated amino acids, such as 4-hydroxyisoleucine, to patients with one or more other antidiabetic agents, in order to obtain an improved (e.g., synergistic or additive) effect. As is discussed further below, examples of agents that can be administered with hydroxylated amino acids, such as 4-hydroxyisoleucine, according to the invention, include insulin, biguanides, sulfonylureas, glinides, glitazones, glucagon like peptide-1 (GLP-1) and agonists thereof, agents that slow carbohydrate absorption, glucagon antagonists, glucokinase activators, and other agents mentioned herein. The methods and compositions of the invention are described in further detail, as follows.

Hydroxylated Amino Acids

Central to the invention is the administration of one or more hydroxylated amino acids (e.g., mono-hydroxylated amino acids, poly-hydroxylated amino acids, or lactonic forms of such hydroxylated amino acids), in combination with one or more other antidiabetic agents, to patients. A specific example of a hydroxylated amino acid that can be used in the invention is 4-hydroxyisoleucine (e.g., the 2S,3R,4S isomer), which has been shown both to stimulate insulin secretion in a glucose dependent manner, and to decrease insulin resistance (see, e.g., U.S. Pat. No. 5,470,879; WO 01/15689; Broca et al., Am. J. Physiol. 277:E617-E623, 1999; the teachings of each of which are incorporated herein by reference).

4-hydroxyisoleucine for use in the invention can be obtained, for example, by chemical synthetic methods. However, this compound is naturally present in high quantities in the seeds of the legume fenugreek (Trigonella foenum-graecum L.), from which it can be purified using methods such as those described in U.S. Pat. No. 5,470,879, WO 97/32577, WO 01/72688, and Wang et al., Eur. J. Org. Chem. 834-839, 2002, the teachings of each of which are incorporated herein by reference. 4-hydroxyisoleucine is preferably administered orally, but also can be administered by other routes including, e.g., subcutaneous, intramuscular, and intravenous routes. The drug can be administered, for example, at a dosage of 0.5 to 200 mg/kg/day. As can be determined by those of skill in this art, the amount of hydroxylated amino acid administered may be decreased when administration is carried out in combination with the use of another antidiabetic agent, as described herein, to obtain an improved (e.g., synergistic or additive) effect.

Examples of agents that can be administered in combination with a hydroxylated amino acid, such as 4-hydroxyisoleucine, according to the invention, are described further below.

Insulin

As is discussed above, type 2 diabetes is characterized by abnormalities in insulin secretion and by insulin resistance of major target tissues, such as muscle, liver, and adipose tissues. This disease has generally been treated by the use of oral antidiabetic agents, such as insulinotropic and insulin sensitizing agents. Type 1 diabetes is characterized by massive destruction of pancreatic β cells, resulting in drastic hypoinsulinemia. Thus, administration of exogenous insulin is central to the treatment of this disease. Insulin resistance also occurs in type 1 diabetes but, in contrast to type 2 diabetes, insulin resistance in type 1 diabetes is not a primary phenomenon but, rather, is a secondary event that can often be reversed by adequate insulin therapy. However, sometimes glycemic control by insulin administration is difficult to achieve, and insulin doses need to be greatly increased. Further, hyperglycemia contributes to impaired insulin action in such subjects.

The binding of insulin to its receptor initiates a signal transduction cascade involving the insulin receptor substrates IRS1, IRS2, etc. A major function of insulin receptor substrates is to activate phosphatidylinositol 3-kinase, which plays a central role in the insulin signaling pathway. Defects in the insulin receptor or in early insulin signaling elements can play an important role in the development of insulin resistance. Indeed, in the case of type 1 diabetes patients with insulin resistance, cellular defects in target tissues have been found that include alterations in insulin binding and intracellular insulin signal transduction involving PI3-kinase activation.

As is discussed above, 4-hydroxyisoleucine is a drug that exhibits both insulinotropic and insulin sensitizing activities. The insulin sensitizing activity of the drug is related to activation of PI3-kinase in muscle and liver. Thus, use of a hydroxylated amino acid (e.g., 4-hydroxyisoleucine) in combination with insulin therapy can lead to increased PI3-kinase activation and thus decreased insulin resistance.

Use of hydroxylated amino acids in combination with insulin therapy can therefore enable the use of decreased doses of insulin. The invention thus includes the use of hydroxylated amino acids, such as 4-hydroxyisoleucine, in the treatment of type 1 diabetes.

Further, the invention also includes approaches involving combining insulin and hydroxylated amino acid therapy with one or more additional therapeutic approaches, such as those described elsewhere herein (e.g., therapy involving the use of one or more biguanides, sulfonylureas, glinides, insulin sensitizing agents (e.g., glitazones), GLP-1 receptor agonists, agents that slow carbohydrate absorption (e.g., acarbose), glucagon antagonists, glucokinase activators, and other agents).

Biguanides

Metformin (Glucophageg, Bristol-Myers Squibb Company, U.S.; Stagid®, Lipha Santé, Europe) is a biguanide compound that is widely used in the treatment of type 2 diabetes. It is the first line drug used in the treatment of obese patients (BMI>27), unless contraindicated by, e.g., impaired renal function. Metformin treatment results in decreased blood glucose levels by several different mechanisms, including reduced intestinal glucose absorption, reduced appetite, enhanced peripheral hepatic utilization (insulin sensitizing effect), and reduced hepatic output. This drug is standardly administered in doses ranging from 500-2550 mg/day, e.g., 850, 1000, 1500, 2000, or 2500 mg, typically taken in one, two, or three doses of, e.g., 500, 850, or 1000 mg each. These amounts may be decreased when used in the combinations of the present invention, as is discussed further elsewhere herein.

The invention includes combination therapy involving the use of a biguanide, such as metformin, with a hydroxylated amino acid, such as 4-hydroxyisoleucine. Also included in the invention are approaches involving the use of biguanides and hydroxylated amino acids (such as 4-hydroxyisoleucine) in combination with other antidiabetic therapies including, for example, those described elsewhere herein (e.g., therapy involving the use of insulin, sulfonylureas, glinides, insulin sensitizing agents (e.g., glitazones), GLP-1 receptor agonists, agents that slow carbohydrate absorption (e.g., acarbose), glucagon antagonists, glucokinase activators, and other agents).

Sulfonylureas and Glinides

Failure to control meal-related glucose peaks is a key factor in the loss of glycemic control in type 2 diabetes. This failure in prandial glycemic control results from an immediate impaired secretory function of pancreatic β cells and from extrapancreatic defects in insulin sensitivity (i.e., insulin resistance). Sulfonylurea drugs, which generally are the first line treatment for non-obese type 2 patients (BMI<27), increase the amount of insulin produced by the pancreas, and thus help to compensate for the body's resistance to insulin. Specific examples of sulfonylurea drugs include gliclazide (Diamicron®), glibenclamide, glipizide (Glucotrol® and Glucotrol XL®, Pfizer), glimepiride (Amaryl®, Aventis), chlorpropamide (e.g., Diabinese®, Pfizer), tolbutamide, and glyburide (e.g., Micronase®, Glynase®, and Diabeta®). As is discussed above, 4-hydroxyisoleucine has insulin stimulatory and insulin sensitizing effects. Thus, combining a hydroxylated amino acid, such as 4-hydroxyisoleucine, with a sulfonylurea drug can be used for meal control in type 2 diabetes.

Treatment with a combination of a hydroxylated amino acid (such as 4-hydroxyisoleucine) and a sulfonylurea drug can be supplemented with treatment employing one or more additional therapeutic agents, such as the antidiabetic agents described herein. For example, one or more of the following types of agents can be used in such combinations: insulin, biguanides, insulin sensitizing agents (e.g., glitazones), GLP-1 receptor agonists, agents that slow carbohydrate absorption (e.g., acarbose), glucagon antagonists, glucokinase activators, and other agents.

Similar to sulfonylureas, meglitinides (i.e., glinides) are drugs that also stimulate the pancreatic β cells to release insulin. As a specific example, repaglinide (Prandin® or NovoNorm®; Novo Nordisk) acts by closing potassium-ATP channels of pancreatic β cells, which results in depolarization of the cell membrane, leading to calcium influx, which in turn triggers insulin secretion. It is fast and short acting, making it a useful pre-meal treatment.

Examples of meglitinide drugs in addition to repaglinide that can be used in the invention include ormitiglinide, nateglinide, senaglinide, and BTS-67582, which can each be taken before meals (also see WO 97/26265, WO 99/03861, and WO 00/37474). Nateglinide (Starlix®) may be particularly useful in reducing post-prandial blood glucose excursions, as it improves first phase insulin secretion.

Treatment with a combination of a hydroxylated amino acid (such as 4-hydroxyisoleucine) and a glinide can be supplemented with treatment employing any combination of the following agents: insulin, biguanides, insulin sensitizing agents (e.g., glitazones), GLP-1 receptor agonists, agents that slow carbohydrate absorption (e.g., acarbose), glucagon antagonists, glucokinase activators, and other agents.

Insulin Sensitizing Agents

As is discussed above, increased levels of glucose and lipids in the blood are fundamental characteristics of diabetes. The resulting glucotoxicity and lipotoxicity can lead to altered β cell function. Glitazones, such as thiazolidinediones, are insulin sensitizing agents and also are effective in reducing free fatty acid and triglyceride concentrations in the blood. As is noted above, 4-hydroxyisoleucine has glucose-dependent insulinotropic activity, as well as extrapancreatic insulin-sensitizing effects. Thus, treatment using a combination of a thiazolidinedione and a hydroxylated amino acid, such as 4-hydroxyisoleucine, has beneficial effects on both glucotoxicity and lipotoxicity.

One example of a thiazolidinedione that can be used in the invention is rosiglitazone maleate (Avandia®, Glaxo Smith Kline). Another example is pioglitazone (Actos®, Eli Lilly, Takeda). Additional examples of thiazolidinedione drugs that can be used in the invention include troglitazone, ciglitazone, isaglitazone, darglitazone, englitazone, CS-011/CI-1037, T 174, and the compounds disclosed in WO 97/41097 (DRF-2344), WO. 97/41119, WO 97/41120, WO 98/45292, and WO 00/41121, the contents of each of which are incorporated herein by reference.

Treatment involving the combined use of a hydroxylated amino acid, such as 4-hydroxyisoleucine, and thiazolidinediones, such as rosiglitazone, can also include other agents, such as insulin, biguanides, sulfonylureas, glinides, other insulin sensitizing agents, GLP-1 receptor agonists, agents that slow carbohydrate absorption (e.g., acarbose), glucagon antagonists, glucokinase activators, and other agents.

Additional examples of insulin sensitizing agents that can be used in combination with a hydroxylated amino acid, according to the invention, include GI 262570, YM-440, MCC-555, JTT-501, AR-H039242, KRP-297, GW-409544, CRE-16336, AR-H049020, LY510929, MBX-102, CLX-0940, GW-501516, and the compounds described in WO 99/19313 (NN622/DRF-2725), WO 00/23415, WO 00/23416, WO 00/23417, WO 00/23425, WO 00/23445, WO 00/23451, WO 00/50414, WO 00/63153, WO 00/63189, WO 00/63190, WO 00/63191, WO 00/63192, WO 00/63193, WO 00/63196, and WO 00/63209, the contents of each of which are incorporated herein by reference.

Glucagon Like Peptide-1 Receptor Agonists

Glucagon-like peptide 1 (GLP-1) is a potent stimulator of glucose-dependent insulin secretion via a cyclic AMP-mediated mechanism in pancreatic β cells. Exendin-4 (1-39) (Ex-4), which is isolated from Gila monster venom, is a highly specific GLP-1 receptor agonist that exhibits a prolonged duration of insulinotropic action. Exenatide® (AC2993; Amylin Pharmaceuticals; Gallwitz et al., Int. J. Clin. Prac. 58(s142):15-19, 2004) is a synthetic version of Ex-4, and has been shown to improve glycemic control by multiple actions, including glucose-dependent stimulation of insulin secretion, suppression of glucagon secretion, slowed gastric emptying, decreased food intake, and reduced weight. Ex-4 has also been reported to increase insulin sensitivity via a PI3 kinase-dependent mechanism. A sustained release formulation (i.e., Exenatide LAR®; Amylin Pharmaceuticals) can also be used. Other examples of GLP-1 agonists that can be used in the invention are described in WO 98/08871 and WO 00/42026, the contents of each of which are incorporated herein by reference.

Treatment involving the combined use of hydroxylated amino acids, such as 4-hydroxyisoleucine, and a glucagon-like peptide 1 receptor agonist, such as Exenatide®, can also include the use of other antidiabetic agents, such as insulin, biguanides, sulfonylureas, glinides, insulin sensitizing agents (e.g., glitazones), agents that slow carbohydrate absorption (e.g., acarbose), glucagon antagonists, glucokinase activators, and other agents.

Agents that Slow Down Carbohydrate Absorption

Agents that slow down carbohydrate absorption can be used to control post-prandial glucose levels. One example of this type of agent is α-glucosidase inhibitors, which act by blocking the breakdown of oligosaccharides and disaccharides from dietary carbohydrates, thus slowing down the absorption of glucose. Examples of α-glucosidase inhibitors include acarbose, miglitol, voglibose, and emiglitate.

Other agents that slow down carbohydrate absorption are those that inhibit gastric emptying. In particular, there are a number of hormones that are known to inhibit gastric emptying, including glucagon like peptide-1, cholescystokinin, and also amylin, which is synthesized and secreted from pancreatic β cells. A synthetic amylin analogue (pramlintide) has been developed for the treatment of diabetes. Use of a combination of a hydroxylated amino acid, such as 4-hydroxyisoleucine, which has insulinotropic and insulin sensitizing properties, and agents slowing down carbohydrate absorption, can be carried out to achieve improved (e.g., synergistic or additive) effects in post-prandial glucose control.

Treatment involving the combined use of hydroxylated amino acids, such as 4-hydroxyisoleucine, and agents that slow down carbohydrate absorption, as described herein, can also include the use of other antidiabetic agents, such as insulin, biguanides, sulfonylureas, glinides, insulin sensitizing agents (e.g., glitazones), GLP-1 receptor agonists, glucagon antagonists, glucokinase activators, and other agents.

Glucagon Antagonists

Glucagon is a hormone that acts in conjunction with insulin to regulate the levels of glucose in the blood. It acts primarily by stimulating cells, such as liver cells, to release glucose when blood glucose levels fall. Thus, to decrease the levels of glucose in the blood in diabetic patients, it is useful to administer glucagon antagonists that, according to the invention, can be administered with a hydroxylated amino acid, such as 4-hydroxyisoleucine.

Examples of glucagon antagonists that can be used in the invention include quinoxaline derivatives (e.g., 2-styryl-3-[3-(dimethylamino)propylmethylamino]-6,7-dichloroquinoxaline; Collins et al., Bioorganic and Medicinal Chemistry Letters 2(9):915-918, 1992); skyrin and skyrin analogues (see, e.g., WO 94/14426), 1-phenyl pyrazole derivatives (U.S. Pat. No. 4,359,474); substituted disilacyclohexanes (U.S. Pat. No. 4,374,130), substituted pyridines and biphenyls (WO 98/04528); substituted pyridyl pyrroles (U.S. Pat. No. 5,776,954); 2,4-diaryl-5-pyridylimidazoles (WO 98/21957, WO 98/22108, WO 98/22109, and U.S. Pat. No. 5,880,139); 2,5-substituted aryl pyrroles (WO 97/16442 and U.S. Pat. No. 5,837,719); substituted pyrimidinone, pyridone, and pyrimidine compounds (WO 98/24780, WO 98/24782, WO 99/24404, and WO 99/32448); 2-(benzimidazol-2-ylthio)-1-(3,4-dihydroxyphenyl)-1-ethanones (Madsen et al., J. Med. Chem. 41:5151-5157, 1998); alkylidene hydrazides (WO 99/01423 and WO 00/39088); and other compounds such as those described in, e.g., WO 00/69810, WO 02/00612, WO 02/40444, WO 02/40445, and WO 02/40446. In addition, further glucagon antagonists can be identified using, e.g., the methods described in U.S. Patent Application Publication US 2003/0138416 A1, the teachings of which are incorporated herein by reference.

Treatment involving the combined use of hydroxylated amino acids, such as 4-hydroxyisoleucine, and a glucagon antagonist, such as those referred to above, can also include the use of other antidiabetic agents, such as insulin, biguanides, sulfonylureas, glinides, insulin sensitizing agents (e.g., glitazones), GLP-1 receptor agonists, agents that slow carbohydrate absorption (e.g., acarbose), glucokinase activators, and other agents.

Glucokinase Activators

Glucokinase is an enzyme that plays a central role in glycolysis, glucose uptake, and glycogen synthesis. Activators of glucokinase have been proposed for use in treating diabetes. Examples of such compounds can be found, for example, in WO 00/58293, WO 01/44216, WO 01/83465, WO 01/83478, WO 01/85706, or WO 01/85707, the contents of each of which are incorporated herein by reference. In addition, further glucokinase activators can be identified using, e.g., the methods described in U.S. Patent Application Publication US 2003/0138416 A1.

Glucokinase activators can be administered with hydroxylated amino acids, such as 4-hydroxyisoleucine, according to the invention, using standard methods. Further, treatment involving the combined use of hydroxylated amino acids, such as 4-hydroxyisoleucine, and glucokinase activators, such as those described in the documents referred to above, can also include the use of other antidiabetic agents, such as insulin, biguanides, sulfonylureas, glinides, insulin sensitizing agents (e.g., glitazones), GLP-1 receptor agonists, agents that slow carbohydrate absorption (e.g., acarbose), glucagon antagonists, and other agents.

Other Agents

Examples of other antidiabetic agents that can be used in combination with a hydroxylated amino acid, such as 4-hydroxyisoleucine (as well as other agents described herein), according to the invention include imidazolines (e.g., efaroxan, idazoxan, phentolamine, and 1-phenyl-2-(imidazolin-2-yl)benzimidazole); glycogen phosphorylase inhibitors (see, e.g., WO 97/09040); oxadiazolidinediones, dipeptidyl peptidase-IV (DPP-IV) inhibitors, protein tyrosine phosphatase (PTPase) inhibitors, inhibitors of hepatic enzymes involved in stimulation of gluconeogenesis and/or glycogenolysis, glucose uptake modulators, glycogen synthase kinase-3 (GSK-3) inhibitors, compounds that modify lipid metabolism (e.g., antihyperlipidemic agents and antilipidemic agents), peroxisome proliferator-activated receptor (PPAR) agonists, and retinoid X receptor (RXR) agonists (e.g., ALRT-268, LG-1268, and LG-1069).

Hyperlipidemia is a primary risk factor for cardiovascular disease, which is particularly prevalent among diabetic patients. Thus, hydroxylated amino acids, such as 4-hydroxyisoleucine, can also be administered, according to the invention, in conjunction with antihyperlipidemic agents or antilipidemic agents (e.g., cholestyramine, colestipol, clofibrate, gemfibrozil, lovastatin, pravastatin, simvastatin, probucol, and dextrothyroxine), optionally, in combination with other agents described herein.

Further, hydroxylated amino acids, such as 4-hydroxyisoleucine, can also be administered, according to the invention, in conjunction with one or more antihypertensive agents (optionally, in combination with other agents described herein), as hypertension has been found to be associated with altered blood insulin levels. Examples of antihypertensive agents that can be used in the invention include β-blockers (e.g., alprenolol, atenolol, timolol, pindolol, propranolol, and metoprolol), angiotensin converting enzyme (ACE) inhibitors (e.g., benazepril, captopril, enalapril, fosinopril, lisinopril, quinapril, and ramipril), calcium channel blockers (e.g., nifedipine, felodipine, nicardipine, isradipine, nimodipine, diltiazem, and verapamil), and α-blockers (e.g., doxazosin, urapidil, prazosin, and terazosin).

Administration

The pharmaceutical agents described herein can be administered separately (e.g., as two pills administered at or about the same time), which may be convenient in the case of drugs that are already commercially available in individual forms. Alternatively, for drug combinations that can be taken at the same time, by the same route (e.g., orally), the drugs can be conveniently formulated to be within the same delivery vehicle (e.g., a tablet, capsule, or other pill). Methods for formulating drugs that can be used in the invention are well known in the art and are described, for example, in Remington: The Science and Practice of Pharmacy (20^{th} edn., A. R. Gennaro, ed.), Lippincott Williams & Wilkins, 2000. These methods include the use of, e.g., capsules, tablets, aerosols, solutions, suspensions, and preparations for topical administration.

Formulations for oral use include tablets containing the active ingredient(s) in a mixture with non-toxic pharmaceutically acceptable excipients. These excipients can be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, and talc). Formulations for oral use can also be provided as chewable tablets, or as hard gelatin capsules in which the active ingredient(s) is mixed with an inert solid diluent, or as soft gelatin capsules in which the active ingredient(s) is mixed with water or an oil medium. Formulations for parenteral administration can contain, for example, excipients, sterile water, or saline; polyalkylene glycols such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Nanoparticulate formulations (e.g., biodegradable nanoparticles, solid lipid nanoparticles, and liposomes) can be used to control the biodistribution of the compounds.

The concentrations of the agents in the formulations will vary, depending on a number of factors including the dosages of the agents to be administered, the route of administration, the nature of the agent, the frequency and mode of administration, the therapy desired, the form in which the agents are administered, the potency of the agents, the sex, age, weight, and general condition of the subject to be treated, the nature and severity of the condition treated, any concomitant diseases to be treated, and other factors that will be apparent to those of skill in the art.

Generally, in the treatment of adult humans, dosages from about 0.001 mg to about 1000 mg (e.g., about 0.05-500, 0.1-250, 0.5-100, 1-50, or 2-25 mg) of each active compound per kg body weight per day can be used. A typical oral dosage can be, for example, in the range of from about 0.001 mg to about 100 mg (e.g., about 0.01-50 or 0.05-10 mg) per kg body weight per day, administered in one or more dosages, such as 1 to 3 dosages. Dosages can be increased or decreased as needed, as can readily be determined by those of skill in the art.

For example, the amount of a particular agent can be decreased when used in combination with another agent, if determined to be appropriate. In addition, reference can be made to standard amounts and approaches that are used to administer the agents mentioned herein. Examples of dosages for drugs mentioned herein are provided in Table 1, below. The drugs can be used in these dosages when combined with a hydroxylated amino acid (e.g., 4-hydroxyisoleucine), which generally is administered in an amount in the range of, for example, 250 mg-1 g/day (e.g., 350-900, 450-800, or 550-700 mg/day). Alternatively, due to the improved (e.g., synergistic or improved) effects obtained when using drug combinations of the invention, the amounts in Table 1 and/or the amount of hydroxylated amino acid administered can be decreased (by, e.g., about 10-70%, 20-60%, 30-50%, or 35-45%), as determined to be appropriate by those of skill in this art.

TABLE 1

| Drug substance | Dosage and/or administration |
|---|---|
| Insulin | 400 IU per vial - 40 IU per day |
| | (mean value) |
| Gliclazide (Diamicron ®) | 80 mg/tablet - 1 to 4 tablets per day |
| Glibenclamide (Daonil ®) | 5 mg/tablet - 1 to 3 tablets per day |
| or Glyburide | (Glibenclamide); |
| (Micronase, Glynase, | 1.25 to 6 mg/tablet - 1 to 2 tablets per |
| Diabeta) | day (Glyburide) |
| Glipizide (Glucotrol ®, | 5 mg/tablet - 1 to 4 tablets per day |
| Glibenese ®) | |
| Glimepiride (Amaryl ®, | 1 to 4 mg/tablet - 6 mg per day maximum |
| Amarel ®) | |
| Chlorpropamide | 250 mg/tablet - 125 to 1000 mg per day |
| (Diabinese ®) | |
| Tolbutamide | 500 mg/tablet - 1 to 4 tablets per day |
| Repaglinide (Prandin ®) | 0.5 to 16 mg per day |
| Nateglinide, Senaglinide | 60 to 120 mg/tablet - 3 tablets per day |
| (Starlix ®) | |
| Tolazamide | 100 to 500 mg/tablet |
| Rosiglitazone | 2 to 8 mg/tablet - 8 mg per day maximum |
| Pioglitazone | 15 to 45 mg/tablet - 15 to 45 mg per day |
| Troglitazone | 200 to 400 mg/tablet - 200 to 600 mg |
| | per day |
| Ciglitazone | 0.1 mg/tablet |
| Exenatide (Amylin) | 0.09 to 0.270 mg per day |
| Acarbose | 50 to 100 mg/tablet - 150 to 600 mg |
| | per day |
| Miglitol | 50 to 100 mg/tablet - 150 to 300 mg |
| | per day |
| Voglibose | 0.1 to 0.9 mg per day |
| Phentolamine | 50 mg 4 to 6 times per day |
| Cholestyramine | 4 g/unit - 12 to 16 g per day |
| (Colestipol) | |
| Clofibrate | 500 mg/capsule - 1 to 4 capsules/day |
| Gemfibrozil (Lipur) | 450 mg/tablet - 2 tablets per day |
| Lovastatin | 10 and 20 mg/tablet |
| Pravastatin | 20 mg/tablet - 10 to 40 mg per day |
| Simvastatin (Zocor ®, | 5 and 20 mg/tablet - 5 to 40 mg per day |
| Lodales) | |
| Probucol | 250 mg/tablet - 1 g per day |
| Dextrothyroxine | 2 to 6 mg per day |
| Alprenolol | 50 mg/tablet - 4 to 8 tablets per day |
| Atenolol | 50 to 100 mg/tablet - 100 to 200 mg |
| | per day |
| Timolol | 10 mg/tablet - 10 to 20 mg per day |
| Pindolol | 5 and 15 mg/tablet - 5 to 60 mg per day |
| Propranolol | 40 mg/tablet - 80 to 160 mg per day |
| Metoprolol | 100 and 200 mg/tablet - 50 to 200 mg |
| | per day |
| Captopril | 25 and 50 mg/tablet - 12.5 to 150 mg |
| | per day |
| Enalapril | 5 and 20 mg/tablet - 5 to 40 mg per day |
| Nifedipine | 10 mg/capsule - 30 to 60 mg per day |
| Diltiazem | 60 mg/tablet - 3 to 6 tablets per day |
| Verapamil | 120 and 240 mg/capsule - 240 to 360 mg |
| | per day |
| Doxazosin | 2 to 8 mg per day |
| Prazozin | 2.5 and 5 mg/tablet - 2.5 to 20 mg |
| | per day |

The invention also provides pharmaceutical compositions including the drug combinations noted above. The drugs can be formulated together in an appropriate form, for example, in a tablet or a capsule. Also included in the invention are kits that include the drug combinations in separate formulations, but with instructions to use them together. The methods, compositions, and kits of the invention can be used in the prevention and treatment of diabetes (types 1 and 2), as well as in the treatment of patients having related conditions, such as pre-diabetes, metabolic syndrome, insulin resistance, and glucose intolerance.

### EXAMPLES

### I. The Combination of 4-hydroxyleucine 2S,3R,4S Isomer with Insulin has an Additive Effect on Glucose Uptake in Differentiated 3T3 Adipocyte Cells

### OBJECTIVE

To determine the effect 4-hydroxyleucine 2S,3R,4S isomer (ID 1101) or insulin, alone and in combination, under various incubation conditions, on the uptake of ³H-deoxy-glucose by differentiated 3T3-L1 adipocyte cells.

### MATERIALS AND METHODS

Briefly, 3T3-L1 adipocyte cells (ATCC; Cl-173) were cultured in 12 well tissue culture plates for 3 days in order to reach confluence (Lakshmanan et al., “Analysis of insulin-stimulated glucose uptake in differentiated 3T3-L1 adipocytes,” Diabetes Mellitus: Methods and Protocols, (Saire Ozcna, Ed.) Humana Press Inc., Tonowa, N.J., 2003, pages 97-103). The culture medium was removed and replaced with differentiation-medium (Green et al., Cell 3:127-133, 1974; Madsen et al., Biochem. J. 375:539-549, 2003), and then the cells were incubated for an additional 9 days. The state of differentiation was confirmed by visual examination. Cell starvation was conducted for 5 hours by replacing the differentiation medium with medium lacking fetal calf serurn. During the starvation period, the cells were exposed ID 1101 (0.5 or 1.0 mM), for 0.5, 1, 2, 4, or 5 hours. The cells were exposed to insulin (0.0167 U/ml; Sigma; Cat. No. 15534) for the last 0.5 hour of the starvation period, either alone or in combination ID 1101. The cells were washed, and then fresh medium containing 16 μM ³H-Deoxy-D-glucose (0.5 μCi/ml) and 10 μM 2-Deoxy-D-glucose was added and the cells were incubated for 10 minutes. Glucose uptake was stopped by washing the cells with ice cold PBS. The cells were lysed and specific activity in the lysate was determined relative to background uptake of ³H-deoxy-glucose. The results were standardized on the basis of protein content per well.

### RESULTS

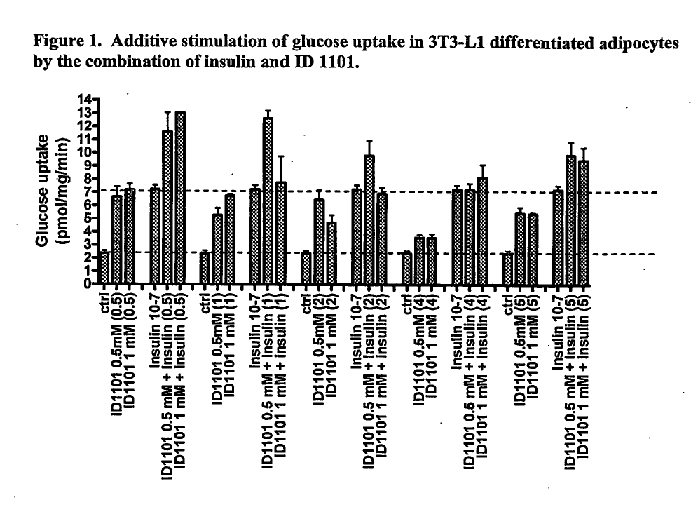

Optimal stimulation of glucose uptake occurred when the cells were exposed for the last 30 minutes of the 5 hour starvation period either to insulin or ID 1101 (0.5 and 1.0 mM) or the combination treatment (FIG. 1). When used as the sole treatment, insulin or ID 1101 (0.5 or 1.0 mM) stimulated glucose uptake by approximately 5 pmol/mg/minute above the background level observed for control cells (2 pmol/mg/minute). However, the combination of insulin with ID 1101, at either 0.5 or 1.0 mM, caused a significant increase in glucose uptake (p<0.05) by approximately 6 pmol/mg/minute over uptake elicited by either of the compounds alone. Glucose uptake was doubled by treating with the combination, indicating that under the conditions tested, the compounds are additive in activity.

### CONCLUSION

Glucose uptake in adipocytes can be stimulated equally by insulin (0.167 U/ml) or ID 1101 (0.5 or 1.0 mM), but when used in combination at these concentrations, an additive effect on glucose uptake is observed.

### II. Effect of 4-Hydroxyisoleucine and Rosiglitazone (Avandia®) Alone and in Combination on Glucose Tolerance in the Diet-Induced Obese C57B6 Mouse

### BACKGROUND

While the mechanism of action remains under investigation, 4-hydroxyisoleucine (ID 1101) has been shown to induce glucose-dependent insulin secretion in vitro and in vivo (Sauvaire et al., Diabetes 47:206-210, 1998) and reduce peripheral insulin resistance (Broca et al., Am. J. Physiol. 277:E617-623, 1999). Rosiglitazone is a Thiazolidinedione that acts by stimulating the peroxisome proliferative-insulin-activating receptors (PPAR), which in turn causes insulin-sensitizing effects on skeletal muscle and adipose tissue (Tiikkainen et al., Diabetes 53:2169-2176, 2004). Hepatic gluconeogenesis also is inhibited. Given the physiological effects of these compounds, it was of interest to determine whether, when used in combination, an additive or synergistic activity might be observed in an animal model of Type 2 diabetes.

### OBJECTIVE

The objective of this study was to determine the effect of Rosiglitazone and ID 1101, alone and in combination, on glucose tolerance in mice rendered hyperglycemic by consuming a high fat diet.

### MATERIALS AND METHODS

C57BL6 mice were received at 7-8 weeks of age and fed a high fat diet (45% of calories from fat) for 8 weeks. Blood glucose was checked and animals with readings between 200 and 220 mg/dL were randomized into control and treatment groups. A group of C57BL6 mice receiving a normal diet was included as a control. Treatment groups included those receiving twice daily treatment by oral gavage with Rosiglitazone (1.5 or 5 mg/kg), ID 1101 (50 or 100 mg/kg), or a combination of Rosiglitazone and ID 1101 (1.5 and 50 mg/kg, respectively).

A baseline oral glucose tolerance test (OGTT) was administered prior to commencement of treatment. The test was repeated on days 7, 14, and 21, to determine whether the treatments influenced glucose tolerance.

### RESULTS

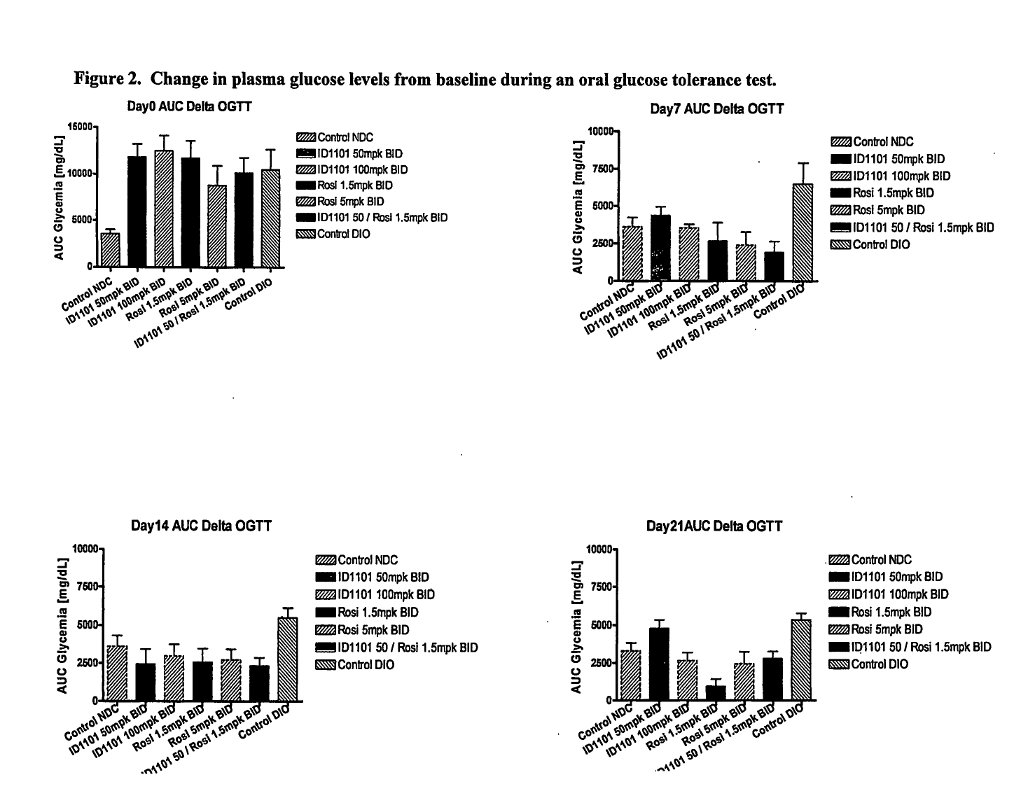

As expected, the baseline OGTT showed that the animals receiving the high fat diet exhibited less tolerance to the glucose challenge than did the normal diet control (NDC.) animals (p<0.05) (FIG. 2). On day 7, the animals underwent an OGTT and the results were compared between groups. The animals treated with the combination of ID 1101 (50 mg/kg) and Rosiglitazone (1.5 mg/kg) were significantly more tolerant to the glucose challenge relative to the high fat diet control animals (DIO) (p<0.05). Similarly, animals treated with Rosiglitazone at 5 mg/kg also were more glucose tolerant that the high fat diet control animals (p<0.05). While there was a trend indicating the drug combination may be more efficacious, the outcome was not statistically significant.

Results of the Day 14 OGT showed a similar but non-significant trend. However, by Day 21, only the mice receiving Rosiglitazone (1.5 or 5 mg/kg) showed significantly improved glucose tolerance relative to the high fat diet control animals (p<0.05)

### CONCLUSION

Only 1 combination of drug concentrations was tested in this study, however the outcome suggests that synergy between the compounds may be observed with different combinations of drug concentrations. Given the toxicity issues associated with Thiazolidinediones, there may be benefit in combining members of this class with ID1101; potentially the dose could be reduced, thus improving safety.

### III. Additive Effect of ID 1101 in Combination with Glibenclamide on Glucose-Dependent Stimulation of Insulin Secretion in INS-1 Cells

### OBJECTIVE

This study was conducted to determine whether ID 1101 in combination with Glibenclamide stimulated insulin secretion to a greater extent than either compound used on its own.

### MATERIALS AND METHODS

The optical isomer 2S,3R,4S of 4-hydroxyisoleucine (ID 1101) was tested in a blinded manner, alone and in combination with Glibenclamide, to determine the insulinotropic effect on INS-1 cells. Briefly, the cells were plated at a density of 2×10⁵ in 12 well plates and incubated for 2 days in RPMI with 10% fetal calf serum and 11 mM glucose. The medium was removed on the third day post-plating and replaced with RPMI containing 3 mM glucose with 10% fetal calf serum. The cells were incubated for an additional 24 hours. On the fourth day post-plating, the medium was removed and replaced with Krebs-Ringers bicarbonate buffer containing 2 mM glucose. The cells were incubated for 30 minutes. The buffer was removed and replaced with Krebs-Ringers bicarbonate buffer with 4.5 mM glucose, containing ID 1101 at 0.1 mM, Glibenclamide alone at 10-10 mM or 10⁻¹¹ mM, or a combination of the 2 compounds. The cells were incubated for 1 hour. Basal insulin secretion was determined by incubating the cells in the presence of buffer with 2 mM glucose. The presence of glucose at 4.5 mM stimulated insulin secretion and served as the positive control.

### RESULTS

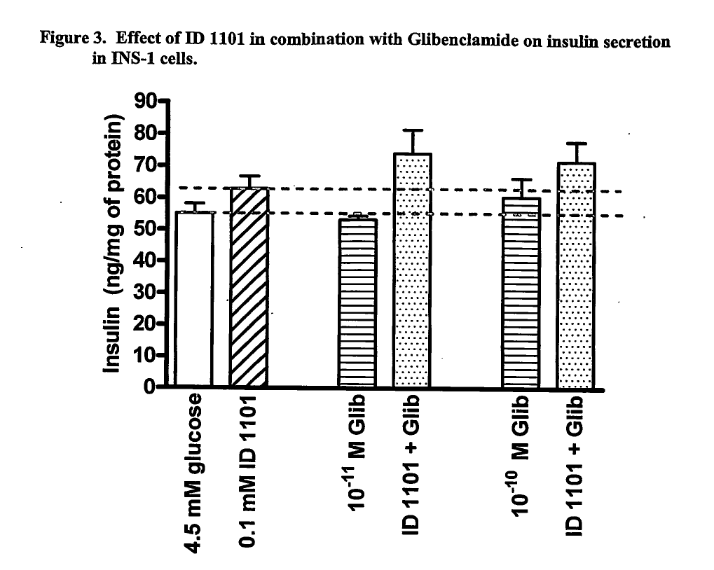

ID 1101 has previously been show to have insulinotropic activity (Broca et al., Eur. J. Pharmacol. 390: 339-345, 2000; Sauvaire et al., Diabetes 47:206-210, 1998) and again stimulated insulin secretion above background levels (FIG. 3). Glibenclamide is a secretagogue and likewise showed a stimulatory effect at 10⁻¹⁰ mM but not at 10⁻¹¹ mM (FIG. 3).

However the combination of ID 1101 at 0.1 mM and Glibenclamide at 10⁻¹¹ mM resulted in a greater stimulatory effect than elicited by either compound alone. The same enhanced stimulatory effect was also observed for the combination with Glibenclamide at 10⁻¹¹ mM.

### CONCLUSION

The combination of Glibenclamide and ID 1101 demonstrates an additive effect on insulin secretion in vitro, using an insulin-secreting cell-line-based screening assay.

### IV. Additive Effect of ID 1101 in Combination with Exendin-4 on Glucose-Dependent Stimulation of Insulin Secretion in INS-1 Cells

### OBJECTIVE

This study was conducted to determine whether ID 1101 in combination with Exendin-4 stimulated insulin secretion to a greater extent than either compound used on its own.

### MATERIALS AND METHODS

The optical isomer 2S,3R,4S of 4-hydroxyisoleucine (ID 1101) was tested alone and in combination with Exendin-4, to determine the insulinotropic effect on INS-1 cells. Briefly, the cells were plated at a density of 2×10⁵ in 12 well plates and incubated for 2 days in RPMI with 10% fetal calf serum and 11 mM glucose. The medium was removed on the third day post-plating and replaced with RPMI containing 3 mM glucose with 10% fetal calf serum. The cells were incubated for an additional 24 hours. On the fourth day post-plating, the medium was removed and replaced with Krebs-Ringers bicarbonate buffer containing 2 mM glucose. The cells were incubated for 30 ninutes. The buffer was removed and replaced with Krebs-Ringers bicarbonate buffer with 4.5 mM glucose, containing ID 1101 at 0.01 or 0.05 mM, Exendin-4 alone at 10⁻⁹ mM or 10⁻¹⁰ mM, or a combination of the 2 compounds. The cells were incubated for 1 hour. Basal insulin secretion was determined by incubating the cells in the presence of buffer with 2 mM glucose. The effect of glucose at 4.5 mM served as the control.

### RESULTS

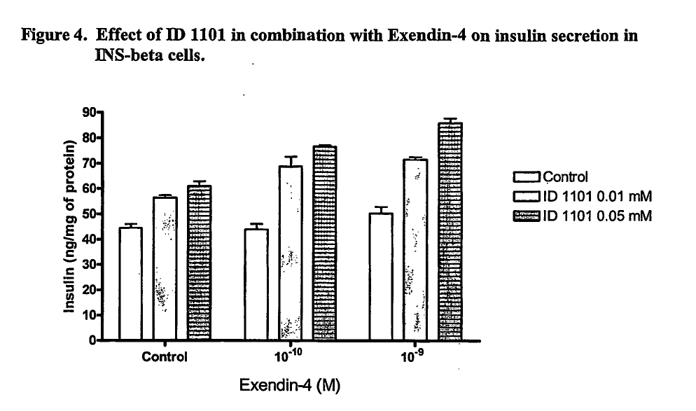

ID 1101 has previously been show to have insulinotropic activity (Broca et al., Eur. J. Pharmacol. 390: 339-345, 2000; Sauvaire et al., Diabetes 47:206-210, 1998) and again stimulated insulin secretion above background levels (FIG. 4). Exendin-4 did not show a stimulatory effect at 10⁻⁹ and 10⁻¹⁰ mM (FIG. 4). However, the combination of ID 1101 at 0.01 and 0.05 mM, and Exendin-4 at either concentration, resulted in a greater stimulatory effect than elicited by either compound alone (p<0.01).

### CONCLUSION

The combination of Exendin-4 and ID 1101 demonstrates an additive effect on insulin secretion in vitro, using an insulin-secreting cell-line-based screening assay.

All publications cited above are incorporated herein by reference in their entirety. Other embodiments are within the following claims.

1-13. (canceled)

14. A pharmaceutical kit comprising 4-hydroxyisoleucine and one or more additional antidiabetic agents selected from the following types of antidiabetic agents: biguanides, sulfonylurea drugs, glinides, insulin-sensitizing agents, glucagon-like peptide 1 receptor agonists, agents that slow carbohydrate absorption, glucagon antagonists, glucokinase activators, imidazolines, glycogen phosphorylase inhibitors, oxadiazolidinediones, dipeptidyl peptidase-IV inhibitors, protein tyrosine phosphatase inhibitors, inhibitors of hepatic enzymes involved in stimulation of gluconeogenesis or glycogenolysis, glucose uptake modulators, glycogen synthase kinase-3 inhibitors, antihyperlipidemic agents, antilipidemic agents, peroxisome proliferator-activated receptor agonists, retinoid X receptor agonists, and antihypertensive agents.

15. The pharmaceutical kit of claim 14, wherein the 4-hydroxyisoleucine is the 2S,3R,4S isomer of 4-hydroxyisoleucine.

16-17. (canceled)

18. The pharmaceutical kit of claim 14, wherein the additional antidiabetic agent is metformin.

19-22. (canceled)

23. The pharmaceutical kit of claim 14, wherein the additional antidiabetic agent is rosiglitazone maleate or pioglitazone.

24. (canceled)

25. The pharmaceutical kit of claim 14, wherein the additional antidiabetic agent is Exenatide®.

26. The pharmaceutical kit of claim 14, wherein the hydroxylated amino acid and the additional antidiabetic agent are formulated into a single composition.

27. The pharmaceutical kit of claim 26, wherein the single composition is a tablet or a capsule.

28. A pharmaceutical composition comprising 4-hydroxyisoleucine, one or more additional antidiabetic agents and a pharmaceutically acceptable excipient, wherein said additional antidiabetic agent(s) is selected from the following types of antidiabetic agents: biguanides, sulfonylurea drugs, glinides, insulin-sensitizing agents, glucagon-like peptide 1 receptor agonists, agents that slow carbohydrate absorption, glucagon antagonists, glucokinase activators, imidazolines, glycogen phosphorylase inhibitors, oxadiazolidinediones, dipeptidyl peptidase-IV inhibitors, protein tyrosine phosphatase inhibitors, inhibitors of hepatic enzymes involved in stimulation of gluconeogenesis or glycogenolysis, glucose uptake modulators, glycogen synthase kinase-3 inhibitors, antihyperlipidemic agents, antilipidemic agents, peroxisome proliferator-activated receptor agonists, retinoid X receptor agonists, and antihypertensive agents.

29. The pharmaceutical composition according to claim 28, for treating diabetes in a patient.

30. A method of treating diabetes in a patient, the method comprising administering to the patient 4-hydroxyisoleucine and one or more additional antidiabetic agents selected from the following types of antidiabetic agents: biguanides, sulfonylurea drugs, glinides, insulin-sensitizing agents, glucagon-like peptide 1 receptor agonists, agents that slow carbohydrate absorption, glucagon antagonists, glucokinase activators, imidazolines, glycogen phosphorylase inhibitors, oxadiazolidinediones, dipeptidyl peptidase-IV inhibitors, protein tyrosine phosphatase inhibitors, inhibitors of hepatic enzymes involved in stimulation of gluconeogenesis or glycogenolysis, glucose uptake modulators, glycogen synthase kinase-3 inhibitors, antihyperlipidemic agents, antilipidemic agents, peroxisome proliferator-activated receptor agonists, retinoid X receptor agonists, and antihypertensive agents.

31. The method of claim 30, wherein the 4-hydroxyisoleucine is the 2S,3R,4S isomer of 4-hydroxyisoleucine.

32. The method of claim 30, further comprising administering insulin to the patient.

33. The method of claim 30, wherein the additional antidiabetic agent is a biguanide.

34. The method of claim 33, wherein the biguanide is metformin.

35. The method of claim 30, wherein the additional antidiabetic agent is a sulfonylurea drug.

36. The method of claim 30, wherein the additional antidiabetic agent is a glinide.

37. The method of claim 30, wherein the additional antidiabetic agent is an insulin-sensitizing agent.

38. The method of claim 37, wherein the insulin-sensitizing agent is a thiazolidinedione.

39. The method of claim 38, wherein the thiazolidinedione is rosiglitazone maleate or pioglitazone.

40. The method of claim 30, wherein the additional antidiabetic agent is a glucagon-like peptide 1 receptor agonist.

41. The method of claim 40, wherein the glucagon-like peptide 1 receptor agonist is Exenatide®.

42. The method of claim 30, wherein the diabetes is type 2 diabetes.

43. The method of claim 30, wherein the hydroxylated amino acid is administered to the patient at or about the same time as the additional antidiabetic agent.
